(19) United States
(12) Patent Application Publication
(10) Pub. No.: US 2006/0246457 A1
(43) Pub. Date: Nov. 2, 2006

(54) Splice site aflp

(75) Inventors: Robert Helene Ghislain Dirks, Gl Oudenbosch (NL); Arie Vogelaar, Ms Dordrecht (NL); Michael Josephus Theresia van Eijk, Ej Herpen (NL); René Cornelis Josephus Hogers, Gr Ede (NL)

Correspondence Address: YOUNG & THOMPSON, 745 SOUTH 23RD STREET, 2ND FLOOR, ARLINGTON, VA, 22202, US

(73) Assignee: KEYGENE N.V., Wageningen (NL)

(21) Appl. No.: 10/563,052

(22) PCT Filed: Jul. 2, 2004
(86) PCT No.: PCT/NL04/00471

§ 371 Date: Apr. 11, 2006

(30) Foreign Application Priority Data
Jul. 2, 2003 (WO) PCT/NL03/00486

(51) Int. Cl. C12Q 1/68; C12P 19/34

(52) U.S. Cl. 435/6; 435/91.2

(57) ABSTRACT

Splice site-specific primers for increasing the informational content of AFLP fingerprints, a method for splice site specific fingerprinting, and a method for targeting genic regions based on conserved splice sites sequences. The invention further describes the conversion into a PCR assay of the splice site-specific fragments obtained by the method of the invention.

### FIELD OF THE INVENTION

The present invention relates to a method for identifying and analyzing nucleic acid sequences that contain or are associated with splice sites. In particular, the invention provides a method for identifying and analyzing nucleic acid sequences based upon polymorphisms associated with such splice sites, a method for targeting genic regions based on conserved splice sites sequences and describes a method for the conversion into a PCR assay of the splice site specific fragments obtained by the method of the invention

### BACKGROUND OF THE INVENTION

Plant breeders undertake continuous efforts to create new varieties that have higher yields and better quality. In many cases, the trait to be improved requires a phase of vegetative growth before the actual assessment or selection can be carried out. For instance, if a breeder wants to select tomato fruits with a better shelf life or higher pigment content, it will take at least two months (from the seedling stage) before the actual observation of fruits can be made. This is of course identical in melon and even true for some agronomic species such as canola. In the last case, oil composition can only be determined when the seeds are ready for harvest. Similar considerations apply to other organisms such as animals, humans etc.

In order to accelerate the identification of suitable lines having the desired characteristics in segregating populations, molecular biologists set up genetic marker systems that allow indirect selection of plants with the desired genetic composition. This means that in an ideal case, DNA from a seedling is analysed to determine whether the desired trait will be present in a much later stage of plant development. The traits envisaged are not only quality traits but also resistances to viruses, fungi etc. In the case of resistance markers, the breeder is much less dependent on disease tests or natural infestations for testing for a successful cross. The criteria that are preferably fulfilled in order to make a genetic marker suitable for the stated purpose is that the DNA sequences that are being visualised are (tightly) linked to the desirable allele and discriminate between, in the case of resistance genes, resistant and susceptible alleles (polymorphisms). Visualisation of the polymorphic DNA sequences can be done using various methods known in the art such as RFLP, AFLP, RAPD, and Microsatellites etc. All these techniques are concerned with the ultimate goal, which is to visualise variation in DNA sequences from any organism that are linked to specific alleles of particular genes. The degree of coupling (association) of the polymorphic DNAs with the alleles of interest determines the suitability (i.e. predictive value for the trait) of the marker. The ultimate marker for a particular gene is the polymorphism within the particular gene locus itself.

As said herein before, various methods for analyzing nucleic acid sequences are known in the art, such as RFLP, AFLP, RAPD, Microsatellites etc. Often, such techniques involve amplification—such as by PCR—of one or more parts of the nucleic acid (s) of a mixture of restriction fragments generated from the nucleic acid (s). The amplified mixture thus obtained is then analysed, e.g. by detection of one or more of the amplified fragments. For example, the amplified fragments may be separated based on differences in length or molecular weight, such as by gel electrophoresis, after which the amplified fragments are visualised, e. g. by autoradiography of the labelled amplified fragments or blotting followed by hybridisation. The resulting pattern of bands is referred to as a (DNA) fingerprint.

Usually in DNA fingerprinting, fingerprints of closely related species, subspecies, varieties, cultivars, races or individuals are compared. Such related fingerprints can be identical or very similar, i. e. contain a large number of corresponding—and therefore less informative—bands. Differences between two related DNA-fingerprints are referred to as genetic markers reflecting DNA-polymorphisms in the genome. These are amplified fragments—i.e. bands—, which are unique in or for a fingerprint and/or for a subset of fingerprints. The presence or absence of such polymorphic fragments in a fingerprint—or the pattern thereof—can be used as a genetic marker. Such a genetic marker can be used, for instance to identify a specific species, subspecies, variety, cultivar, race or individual; to establish the presence or absence of a specific inheritable trait and/or of a specific gene; and/or to determine the state of a disease. For a further discussion of DNA-fingerprinting, DNA-polymorphisms, genotyping, PCR and similar amplification techniques, as well as the techniques and materials used therein, reference is inter alia made to the prior art mentioned hereinbelow, as well as to the standard handbooks.

One DNA-fingerprinting technique—which is advantageous in that it requires no prior knowledge of the sequence to be analysed—is selective restriction fragment amplification or AFLP. In general, AFLP comprises the steps of:
(a) digesting a nucleic acid, in particular a DNA or cDNA, with one or more specific restriction endonucleases, to fragment the DNA into a corresponding series of restriction fragments;
(b) ligating the restriction fragments thus obtained with a double-stranded synthetic oligonucleotide adapter, one end of which is compatible with one or both of the ends of the restriction fragments, to thereby produce tagged restriction fragments of the starting DNA;
(c) contacting the tagged restriction fragments under hybridizing conditions with one or more oligonucleotide primers;
(d) amplifying the tagged restriction fragment hybridised with the primers by PCR or a similar technique so as to cause further elongation of the hybridised primers along the restriction fragments of the starting DNA to which the primers hybridised; and
(e) detecting, identifying or recovering the amplified or elongated DNA fragment thus obtained.

The AFLP-fingerprint thus obtained provides information on sequence variation in (subsets of) the restriction enzyme sites used for preparation of the AFLP template and the nucleotide (s) immediately adjacent to these restriction enzyme sites in the starting DNA.

By comparing AFLP-fingerprints from related individuals, again polymorphic fragments (also referred to as AFLP-markers) can be detected/identified, e.g. for the purposes mentioned hereinabove.

For a further description of AFLP, its advantages, its embodiments, as well as the techniques, enzymes, adapters, primers and further compounds and tools used therein, reference is made to U.S. Pat. No. 6,045,994, EP-B-0 534 858, EP 976835 and EP 974672, WO01/88189 and Vos et al. Nucleic Acids Research, 1995, 23, 4407-4414.

Although the basic AFLP technology (basic AFLP) is a very efficient technique for identifying and analyzing polymorphisms in random subsets of nucleic acid sequences, DNA or cDNA, basic AFLP does not contain any discriminating factor that allows the amplification of selected subsets with particular sequence characteristics. For instance, basic AFLP cannot distinguish between coding and non-coding regions. Basic AFLP is also not specifically capable of identifying AFLP markers that are associated with specific or predefined sections of the genome such as those representing genic regions of the genome (intron and exon sequences).

One of the objects of the present invention is to provide methods for the identification and/or analysis of nucleic acid sequences. It is also an object of the present invention to provide for improved or alternative methods for the identification or analysis of splice site based or splice site located polymorphisms, and to identify and analyze markers based thereon. It is a particular object of the present invention to provide for such a method involving the use of (parts of) AFLP technology.

### DESCRIPTION OF THE INVENTION

The present invention pertains to a method for the analysis of polymorphisms. The method provides for the identification or determination of polymorphisms that may be enriched for sites carrying or encoding for splice site sequences in the genome under investigation. The method is directed to providing polymorphisms and fingerprints that are more closely linked to, or are at least indicative of, genes, in particular to polymorphisms and fingerprints that are more linked to the presence of introns and exons. The method is based on the structural features of the nucleotide sequence at the intron-exon boundaries. The method is also based on (parts of) the AFLP technology

Generally, the above-described objects of the invention are achieved by a method wherein the nucleic acid is analysed, and more in particular one or more adapter-ligated restriction fragments derived from the nucleic acid are analysed, using at least one primer (depicted herein as splice site primer, splice site-specific primer or S3P primer). The S3P primer preferably targets splice site borders (intron-exon junctions, splicing junctions, intron-exon boundaries) and is preferentially designed to hybridise to (and prime extension from) conserved splice site sequence motifs present in target nucleic acids. Thus, the above objects are achieved by amplifying the nucleic acid—and in particular one or more adapter-ligated restriction fragments generated from the nucleic acid—with at least one S3P-primer and then analysing the amplified mixture thus obtained, which is enriched for genic sequences/fragments.

### DETAILED DESCRIPTION OF THE INVENTION

In its broadest scope, the invention comprises the use of at least one S3P-primer in analyzing nucleic acid sequences. In particular, the invention comprises the use of a S3P-primer in combination with another primer in analyzing nucleic acid sequences or of the use of two primers in analyzing nucleic acid sequences of which one primer is an S3P primer. More in particular, the invention comprises the use of a S3P-primer in analysing a nucleic acid sequence for (the presence or absence of) splice site-associated polymorphisms/markers.

By splice site-associated polymorphism or marker is generally meant any polymorphism or marker that is caused by, and/or that is related to, the presence and/or absence of a splice site in the nucleic acid, e.g. at one or more specific sites in the nucleic acid. Under splice site-associated polymorphism in the present invention is also understood the polymorphism associated with the AFLP technology. In AFLP, the polymorphism is mostly located in the recognition sites of the restriction endonuclease(s). Thus a polymorphic fragment obtained by the use of an S3P primer and an AFLP primer is considered a splice site-associated polymorphic fragment, regardless of the location of the polymorphism (in the splice site or the recognition sites of the restriction endonuclease used). Usually, in the invention, the presence or absence, respectively, of a polymorphism at such site (s) in the nucleic acid to be analysed (or for instance the presence of a different splice site at such site (s) will lead to the generation of different polymorphic fragments, for instance bands that correspond to amplified fragments of different size and/or length (so-called fragment length polymorphisms).

The coding sequences (exons) of genes are frequently interrupted by non-coding stretches of DNA (introns). Introns are generally transcribed as part of precursor RNAs and subsequently removed by a cleavage-ligation process called splicing. The structural features of introns and the underlying mechanism for splicing form the basis for a classification for different kinds of introns. The structural features for accurate splicing are also found at the borders between introns and exons (the junctions). The junctions have well conserved, though relatively short, consensus sequence. It is possible to assign a specific end to every intron by relying on the conservation of intron-exon junctions. The junctions can be aligned to conform to the consensus sequence given in FIG. 2A. The subscript in FIG. 2A indicates the percent occurrence of the specified base (or type of base (N=A,C,T,G, Py=pyrimidine base, Pu=purine base) at each consensus position among a large number of introns analysed. High conservation is found only immediate within the intron at the presumed junctions. This identifies the sequence of a generic intron as GT . . . AG. Because the intron defined in this way starts with the dinucleotide GT and ends with the dinucleotide AG, junctions are often described as conforming to the GT-AG rule (the actual sequences in the RNA are, of course, GU-AG). Note that the two sites have different sequences and so they define the ends of the intron directionally. They are generally named proceeding from the left to the right along the intron, that is, as the left (or 5′) and right (or 3′) splice sites. Sometimes they are called donor and acceptor sites. The consensus sites are implicated as the sites recognised in splicing by point mutations that prevent splicing in vivo and in vitro. The GT-AG rule describes the splice sites of nuclear genes of many (if not all) eukaryotes. This implies that there is a common mechanism for splicing intron out of RNA. For introns of mitochondria, chloroplasts and other organelles, other consensus sequences are known and can be applied likewise in the present invention.

Accordingly, as visualised in FIG. 2, introns usually contain at one end (the 5′ end here) the dinucleotide GT and at the distal (the 3′ end here) end the dinucleotide AG. This is the so-called GT-AG rule (see Lewin, Genes VI, Oxford university press 1998, ISBN 0 19 8577788 pp. 885-920; Lewin, Genes IV, Oxford university press 1990, ISBN 0 0198542682 page 578-609) The GT-AG rule describes the splice sites of nuclei genes of many eukaryotes. On the 5′ end of the intron (thus in the exon), 2 nucleotides are also highly conserved, in many cases 5′-AG-3′. On the 3′-side of the GT dinucleotide (thus in the intron) high conservation can be seen for a tetranucleotide 5′-AAGT-3′. Taken together this means that at the 5′side of the intron and extending two nucleotides into the exon eight nucleotides can be identified. These eight nucleotides can be identified with high homology throughout eukaryotes. It is expected in the art that within the different kingdoms and in particular at the level of species, the degree of conservation will be very high. At the 3′ border (in the intron), sequence conservation is also observed (see Lewin 1998, 1990) and similar observations apply to the 3′ end of the intron exon junction.

Using these consensus sequences of intron exon junctions, primers can be designed that allow for the selective amplification of fragments that contain these consensus sequences. This allows the selective amplifications of fragments that contain splice site junctions and thus for the selective amplification of gene related fragments. The subset of selectively amplified fragments hence comprises an increased amount of information on markers that are directly related to genes. This is in contrast with the conventional technologies such as RAPD, AFLP wherein primarily anonymous (non-targeted) sequences and not the gene or allele of interest itself.

Various types of introns result in different splice sites having different consensus sequences. Examples thereof are those described in Singer & Berg, Genes and Genomes, 1991, University Science Books ISBN 063202879-6 pp 556 ff.; T. Cech, Cell 44 (1986), 207; T. Cech, Int. Rev. of Cytology 93 (1985) 3; CR Cantor and CL Smith, Genomics, John Wiley & Sons, New York, pp. 530 ff.; T. Brown, Genomes, 1999, Bios Scientific publishers ISBN 1859962017, pp. 212-219. Based on the generalised knowledge available in these citations as well as in numerous other publications describing splice site structures and consensus structures, the skilled man can design a suitable splice site-specific primer that can be used in the present invention. For the identification of putative splice sites in sequences, there are also various computer programs available such as from NetPLAntgene [http://www.cbs.dtu.dk/services/NetPGene]; BDGP [http://www.fruitfly.org/seq_tools/splice.html]; and Genio [http://genio.informatik.uni-stuttgart.de/GENIO/splice/].

Table 1 discloses various types of splice sites with locations and/or consensus sequences. The ˆ depicts a splice site, W=A or T; M=A or C; R=A or G; Y=C or T; K=G or T; S=G or C; H=A, C or T; B=C, G or T; V=A, C or G; D=A, G or T; N=A, C, G or T and n indicates multiple nucleotides. This nucleotide nomenclature is used throughout the application. Invariant bases (consensus sequence) are underlined. The bases are shown as they occur in RNA.

TABLE 1
Splice sites:

| Intron | 5′ splice | Near 3′ splice | 3′ splice | |
|---|---|---|---|---|
| type | junction | junction | junction | Where found |
| GU-AG | CRG\hat{ }GU(A/G) | A | YnAG\hat{ }N | Nuclear Pre- |
| | AGU | | | mRNA |
| | | | | (general) |
| GU-AG | \hat{ }GUAUGU | UACUAAC | YnAG\hat{ }N | Nuclear Pre- |
| | | | | mRNA (yeast) |
| TRNA | N\hat{ }N | | N\hat{ }N | |
| Group I | U\hat{ } | | G\hat{ } | Nuclear rRNA, |
| | | | | mitochondrial |
| | | | | mRNA and |
| | | | | rRNA, |
| | | | | organelle |
| | | | | RNAs, bacterial |
| | | | | RNAs |
| Group II | \hat{ }GUGCG | | YnAU\hat{ } | Mitochondrial |
| | | | | mRNA, |
| | | | | organelle |
| | | | | RNAs, |
| | | | | prokaryotes |
| Group III | | | | organelle RNAs |
| Chloroplast | \hat{ }GUG(C/U)G | | YnAU\hat{ } | |
| mRNA | | | | |
| (Euglena) | | | | |
| Pre-tRNA | | | | Eukaryotic |
| introns | | | | nuclear Pre- |
| | | | | TRNA |
| Archael | | | | Various RNAs |
| introns | | | | |

Other introns such as twintrons (i.e. introns within introns, such as described in D. W. Copertino and R. B. Hallick, “Group II and group III introns of twintrons: potential relationships to nuclear pre-mRNA introns”. TIBS (1993) 18:467-471; or. R. G. Drager and R. B. Hallick, “A complex twintron is excised as four individual introns”. Nucl. Acids Res. (1993) 21:2389-2394 are also within the scope of the invention.

The method of the invention is schematically illustrated in the non-limiting FIG. 1. In FIG. 1, the S3P-primer is indicated as (1), the nucleic acid to be amplified—also referred to hereinbelow as the target DNA—is indicated as (2). The (sequence of) a splice site present in/on the target DNA is indicated as (3), with the intron part of the splice site as (3A) and the exon part of the splice site as (3B). As schematically shown in FIG. 1, the S3P-primer (1) is (intended to be) complementary to that part of the sequence of the target DNA (2) that at least comprises a part of the splice site sequence (3). Preferably the S3P-primer comprises the junction of the splice site (between 3A and 3B), so as to allow—e. g. during amplification—the extension of the S3P-primer (1) in the 3′-direction along the target DNA (2), which serves as a template for the extension of the S3P-primer (1). As also schematically shown in FIG. 1, the S3P-primer (1) may be considered to comprise essentially two parts, i.e. a 3′-part and a 5′-part, indicated in FIG. 1 as (4) and (5), respectively. Part of the 3′-part (4) of the S3P-primer (1) is (intended to be) essentially complementary to (part of the sequence of) the intron (3A), more in particular to part of the consensus sequence of the intron section. The 5′-part (5) of the S3P-primer (1) is (intended to be) complementary to the exon, more in particular to the consensus sequence of the exon sequence (3B).

The S3P-primer (1) will be at least such that, when (the sequence of) a splice site (3) to which the S3P-primer (1) is complementary is present in the target nucleic acid, it is capable of hybridizing with the splice site (3) so as to allow extension of the S3P-primer (1) along the target nucleic acid (2). The skilled person will understand that the S3P primer comprises sequence that may be complementary to splice site sequences either in the sense strand or in the non-sense strand. Usually, a S3P-primer (l) used in the invention will contain a total of between 8 and 20 nucleotides, and in particular between 12 and 16 nucleotides. Of these, between 2 and 10 nucleotides, and in particular between 4 and 8 nucleotides, preferably between 5 and 7 nucleotides will form part of the 3′-part (4) of the S3P-primer (1) i.e. the part that is complementary to (the intron-derived motif of) the splice site. Between 4 and 10 nucleotides, and in particular between 6 and 8 nucleotides will form part of the 5′-part (5) of the S3P-primer (1), i.e. the exon region. The S3P primer is a primer comprising a conserved splice site border sequence or at least part of a consensus sequence of a splice site border sequence, preferably at least 50% of the consensus sequence, more preferably at least 60%, 70%, 80%, 90%, in particular 95% and most preferably 100% of the consensus sequence. In one embodiment the splice site specific or S3P primer is a primer that comprises a section that is derived from the GT . . . AG consensus motif of the intron and is capable of annealing to part of the GT . . . AG consensus motif, preferably in combination with one or more of the consensus nucleotides of the exon, as depicted in FIG. 2A. In a preferred embodiment, the S3P primer comprises at least the oligonucleotide fragment GT. More preferably, the S3P primer comprises at least the oligonucleotide fragment: X₁X₂GT wherein X stands for A, C, T, or G. Preferably X₁ is A. Preferably X₂ is G. In a further preferred embodiment the S3P primer comprises at least the oligonucleotide fragment: GTX₃X₄X₅X₆ wherein X stands for A, C, T, or G. Preferably X₃ is A. Preferably X₄ is A. Preferably X₅ is G. Preferably X₆ is T.

In a more preferred embodiment the primer comprises the oligonucleotide fragment X₁X₂GTX₃X₄X₅X₆. It is noted that X₁ through X₆ (also depicted as the variable nucleotides) can be selected independently from each other, thus a primer comprising the fragment ANGTNNNT is within the scope of the present invention. A preferred splice site primer contains at least 4 nucleotides selected from amongst the generalised consensus sequence AGGTAAGT, more preferably 5 nucleotides, particularly preferred 6 nucleotides, more particularly preferred 7 nucleotides. This means that a primer according to this embodiment can comprise, for example, the following structure: ANGTNNGT or NNGTNNGT. Most particularly preferred is that the primer contains the generalised consensus sequence AGGTAAGT.

Based on the guidelines for primer design and variations therein as outlined herein above, the skilled man can design splice site specific primers for splice sites having other consensus sequences, based for instance on the consensus sequences outlined in Table 1 or otherwise identified in the literature.

As the splice sites may be present in a degenerated form in a genome, it may be useful to use a set of S3P primers. Such a set comprises more than one S3P primer. Preferably, a set comprises two, three, four, five, six, seven, eight, nine or ten different S3P primers. Preferably, such a set comprises more than 10, 20, 30, 40 or even more than 50 different S3P primers. Each primer in a set differs in at least one, preferably two more preferably three and most preferably four or more of its nucleotides from the other primers in the set. Each of the primers in the set follows the general structure for S3P primers as outlined herein before. Such a set is advantageously if, for instance for a certain species the consensus sequence is not known or contains more variation than usual.

Within such a set of S3P primers, the primers can be independently varied and the variable nucleotides can be selected at will. Preferably, the variation within the S3P primer set can be provided by variation of X₁-X₆ between two primers in the set. For example, for a first primer in the set X₁ is A and for the second primer in the set X₁ is T, where the remaining sequence of the primer is identical for the first and second primer.

One of the preferred splicing sites of the present invention contains an average structure that can be depicted as A(64)G(78) in the exon; G(100)T(100)A(62)A(68)G(84)T(63) on the 5′ end of the intron; 12PyNC(65)A(100)G(100) on the 3′ end of the intron. The numerical values indicate the percent occurrence of the specified base or type of base at each consensus position of the splice site. This means that throughout a set of splice sites, whether from different organisms or genomes, the consensus sequence of the splice site is a statistical average. The value 100 means that each splice site contains that nucleotide, or in other words, that specific nucleotide has an occurrence of 100% at that positioning this type of splice site. A value less than 100%, for instance 62% means that there is variation at that position in that type of splice site amongst a wide number of splice sites of that type that have been investigated. From that statistical average, the most common nucleotide has an occurrence of 62%. The complementary percentage is distributed amongst the other nucleotides. Thus, A(62) means that, on average, 62% of the splice sites in contains an A at that particular position of the splice site. The other 38% can be distributed amongst C, G or T. The percentages may differ when specific genomes are targeted and can be adjusted accordingly. Sets of S3P primers may be synthesised that are based on the above average structures of splice sites. Thus a preferred set of S3P primers based on the average 5′ intron structure may have a composition whereby in the first (i.e. most 5′) position of the section that corresponds to the splice site sequences in the primers in the set 64% of the nucleotides are A and 12% of the nucleotides are G, 12% of the nucleotides are T, 12% of the nucleotides are C, in the second position 78% of the nucleotides are G, 7.33% of the nucleotides are A, 7.33% of the nucleotides are T and 7.33% of the nucleotides are C and so on for the further positions in the S3P primer set. A preferred set of S3P primers based on the average 3′ intron structure thus may have a composition whereby in the first 12 (i.e. most 5′) positions of the section that corresponds to the splice site sequences in the primers in the set a 50/50 mixture of pyrimidines is present, in the 13^{th} position equal amounts of A, G, C and T are present, in the 14^{th} position 65% of the nucleotides are C and 11.66% of the nucleotides are A, 11.66% of the nucleotides are G and 11.66% of the nucleotides are C, and so on. In these composition of S3P primers the percentages indicated above are only used as examples and may be adapted by the skilled person, in particular the percentages of the three different minority nucleotides are not necessarily the same. A preferred S3P primers based on the average 3′ intron structure has a composition whereby in the first 12 positions nucleotide analogues are present that contain degenerate bases mimicking pyrimidines, i.e. a C/T mix, or mimicking purines, i.e. an A/G mix, in its complement. Such nucleotide analogues contains e.g. the P and K bases (mimicking respectively pyrimidines and purines) as described by Kong and Brown (1989, Nucl. Acids Res 17: 10373-383; 1992, Nucl. Acids Res 20: 5149-52). The length of the sections in the S3P primers having the sequence based on the average 5′ and 3′ splice site structures or their complements are as described above. It is preferred that when a set of S3P primers is designed, the average composition of the splice site specific parts of the primers corresponds to this distribution, or at least for 70, 80 or 90%. It is noted that when other splice sites are targeted similar sets of primers can be designed taking into account the average composition of the splice sites throughout a genome of interest.

It is noted that the S3P primer of the present invention may comprise other nucleotides than the nucleotides that are part of the consensus sequence of the splice site. These nucleotides can be located at any position in the S3P primer (and not just at the X₁-X₆ positions) that does not form a part of the GT or AG consensus sequence such as the 3′ end or at the 5′ end or between sections of the consensus sequence. Alternatively, one or more of the nucleotides X₁₋₆ of the consensus sequence may be replaced by so called universal nucleotide analogues such as inosine, and/or they may contain LNAs, PNAs etc.

The splice site can be approached by two routes, i.e. orientations, (exon-to-intron or intron-to-exon) and two different primers can be designed accordingly. The skilled person will know that in the exon-to intron orientation the S3P primer has a sequence that is complementary to the sense strand of the splice site sequence and that in the intron-to-exon orientation the S3P primer has a sequence that is complementary to the nonsense strand of the splice site sequence. The use of these two different primers may lead to different (fingerprinting) results and hence to the determination or identification of different polymorphisms. Both types of primers (exon-to-intron or intron-to-exon) may be present in a set of S3P primers.

The S3P primer can be elongated by conventional elongation techniques that may lead to linear or exponential amplification of the restriction fragment. Examples thereof are Strand Displacement Amplification (SDA), etc. Preferably, an exponential amplification technology is used. In particular, an amplification technique based on the polymerase chain reaction (PCR) is used. PCR commonly employs at least two primers. In the present invention, this means that, when PCR is used, additional to the S3P primer a second or further primer is used. The second or further primer may be a random primer or a primer that is directed against a specific target sequence such as a (retro)transposon, an NBS region, a microsatellite, or a second splice site. Alternatively, the primer can be directed against other conserved sequences present in the intron. An example thereof is the conserved region where, during the transesterification reaction the hydroxyl attached to the 2′ carbon of the adenosine promotes the reaction to form the lariat structure. In yeast, such conserved regions are known as TACTAAC regions. Alternatively, the primer can be directed against the adapter of adapter-ligated restriction fragments such as an AFLP primer. Preferably, the second primer is a second S3P primer or an AFLP primer, more preferably an S3P primer.

In an alternative embodiment of the invention, one or more of the primer used to analyze the nucleic acid sequence(s) can be associated with or is directed against specific target sequence such as a (retro)transposon or an NBS region. This primer can be combined with one or more of AFLP primers, S3P primers, or random primers.

In a particular preferred embodiment of the present invention, the second primer used in the PCR amplification is an AFLP primer. The combination of a S3P primer with an AFLP primer as the second primer is used here to illustrate the principle of the invention. It is explicitly noted that as the second primer any of the abovementioned second primers can be used without departing from the gist of the invention. The prior art does not describe or suggest a method for analyzing splice site associated polymorphisms or markers involving the use of both a S3P-primer and an AFLP-primer.

The AFLP-primer used in the invention, indicated as (7) in FIG. 1, is essentially the same as a conventional AFLP-primer, in that it is (at least) complementary to (the sequence of) an adapter, indicated as (8) in FIG. 1, that has been linked to the target DNA (2), so as to allow—e. g. during amplification—the extension of the AFLP-primer (7) in the 3′-direction along the target DNA (3), which serves as a template for the extension of the AFLP-primer (7). Most preferably, as in AFLP, the primer contains, at its 3′-end, a number of so-called selective bases/nucleotides—indicated as (9) in FIG. 1—that are (intended to be) complementary to (same number of) bases/nucleotides that, in the target DNA (2), are directly adjacent to the 3′ end of the adapter (8). Using the S3P-primer (1) and the AFLP-primer (7), the target nucleic acid (2) is amplified, e. g. as indicated by the arrows in FIG. 1. In particular, during the amplification, the S3P-primer (1) will be extended along one strand of the (double stranded) target DNA (2) and the AFLP-primer will be extended along the other strand of the (double stranded) target DNA (2), e. g. so as to allow for efficient/exponential amplification. The mixture of amplified products/fragments thus obtained may then be analysed, e. g. by detecting/visualizing at least one, and up to essentially all, of the amplified products/fragments, e. g. as further described hereinbelow.

In one aspect, the invention relates to the use of (the combination of) an S3P-primer and an AFLP-primer in amplifying a nucleic acid sequence (herein also referred to as the target nucleic acid). In this aspect of the invention, and with reference to FIG. 1, the target nucleic acid usually will comprise, but is not limited to an adapter (8) and a further nucleic acid sequence, indicated as (11) in FIG. 1, to which the adapter has been ligated. In particular, in this aspect of the invention, the further nucleic acid sequence (11) present in the target nucleic acid (2) may be a restriction fragment. For instance, the further nucleic acid (11) may be a restriction fragment derived from a starting DNA—including but not limited to genomic DNA, or recombinant DNA such BAC DNA, cosmid DNA or plasmid DNA—by restriction with a restriction endonuclease (as further described hereinbelow), although the invention in its broadest sense is not limited thereto. In addition, the target nucleic acid (2) will usually be a DNA sequence, and in particular, a double stranded DNA sequence, although the invention in its broadest sense is again not limited thereto.

The target nucleic acid (2) may comprise a single adapter (8) but usually comprises two adapters (8), e.g. each ligated to one end of the restriction fragment (11) present in the target nucleic acid. In addition, when two adapters (8) are present, they may be the same or different.

The target nucleic acid (2) may be part of a mixture of such target nucleic acids. For instance, when the target nucleic acid comprises a restriction fragment ligated to an adapter, it may be part of a mixture of such adapter-ligated restriction fragments. Such a mixture may for instance be obtained by ligating a adapter to a mixture of restriction fragments, which may be carried out in a manner known per se, for instance as described in the prior art, including but not limited to EP 0 534 858.

Optionally, such a mixture of target nucleic acids may (already) have been subjected to a (pre) amplification step, i. e. prior to the amplification with the S3P-primer and the AFLP-primer. For instance, when the target nucleic acid (s) (2) contain two adapters (8), such a pre-amplification may be carried out as a conventional AFLP-pre-amplification i.e. using +0/+0 AFLP primers, for which reference is made to EP 0 534 858 and Vos et al., cited herein. This pre-amplification may also have been a selective pre-amplification for reducing the complexity of the mixture i.e. using +n+m AFLP primers, wherein n, m are integers, independently ranging from 1 to 10.

The target nucleic acid (2) preferably also comprises (or is at least suspected to contain) at least one splice site, or otherwise the target nucleic is at least part of a mixture of such target nucleic acids of which a target nucleic acid comprises (or is suspected to contain) a splice site; and in particular a splice site to which the S3P-primer (1) can and/or is intended to hybridise.

The AFLP-primer (7) will be essentially the same as a conventional AFLP primer, e.g. as described in EP 0 534 858, and will generally contain a constant region indicated as (10) in FIG. 1—and one or more selective nucleotides in a selective region (9) at the 3′-end thereof. In addition, the AFLP-primer (7) is most preferably essentially complementary to at least one of the adapters (8) used, e. g. so as to allow extension of the AFLP-primer (7) along the target nucleic acid (2). Preferably, the AFLP-primer (7) will contain a total of between 15 and 50 nucleotides, and in particular between 18 and 30 nucleotides. Also, preferably, the AFLP-primer (7) will contain between 0 and 6, preferably 1 or 2 or 3 or 4 selective nucleotides.

The amplification of the target nucleic acid (2) with the S3P-primer (1) and the AFLP-primer (7) may be carried out under conditions known per se, including but not limited to conditions known per se for amplifications in general or using conditions known per se for amplification using AFLP-primers. Such conditions are for instance described in the above-mentioned prior art (e.g. EP 0 534 858 for AFLP-primers) and some non-limiting examples of suitable conditions are given in the Experimental Part hereinbelow. It is envisaged that based upon these disclosures, the skilled person will be able to select (a range of) optimal conditions for the amplification of a given (mixture of) target nucleic acid (s) with a given combination of S3P-primer and AFLP-primer.

Preferably, the amplification is carried out using only one S3P-primer as described above and only one AFLP-primer as described above, although the invention in its broadest sense is not limited thereto.

In addition, the S3P-primer and the AFLP-primer are preferably such that they allow for efficient/exponential amplification. In this respect, it should be noted that, when the target nucleic acid is part of a mixture of such target nucleic acids, in the amplification step usually more than one of the target nucleic acids that are present is the mixture will be amplified, i. e. to provide a mixture of amplified fragments.

After the amplification with the S3P-primer and the AFLP-primer, the amplified nucleic acid thus generated is detected. For instance, when the amplification step has provided a mixture of amplified fragments as described hereinabove, one or more-and up to essentially all-amplified fragments present in the mixture may be detected. The detection may be carried out using any technique known per se for the detection of an amplified nucleic acid/fragment and/or for analyzing a mixture of amplified nucleic acids/fragments. Suitable techniques are described in the abovementioned art and for instance include techniques in which the amplified fragments are separated and visualised (e. g. (capillary) gel electrophoresis and autoradiography to provide a fingerprint); (other) detection techniques based upon the mass and/or the size of the amplified fragments; and techniques involving the hybridisation of one or more of the amplified fragments to a complementary nucleotide sequence (in which the complementary nucleotide sequence may for instance be immobilised on a suitable carrier, e. g. as part of an array of such nucleotide sequences) followed by detection of such hybridisation events. Generally, these detection techniques will be such that they allow for the detection of polymorphisms, as further described below. The invention is not limited to the use of one primer that selectively amplifies ‘gene-lie’ sequences by targeting to intron-exon junctions and a AFLP primer, but may also be by using two splice site specific primers.

In another aspect, the invention relates to a method for analyzing a nucleic acid sequence, the method at least comprising the steps of: (a) amplifying a restriction fragment generated from the nucleic acid to be analysed, in which the restriction fragment has been ligated to a adapter, with one or more S3P-primers and/or an optional AFLP-primer to provide an amplified nucleic acid sequence; and optionally comprising the further step of: (b) detecting at least one of the amplified nucleic acid sequences thus obtained.

More specifically, this aspect of the invention relates to a method for analyzing a nucleic acid sequence, the method comprising the steps of:
(a) restricting the starting nucleic acid with a restriction endonuclease to provide a mixture of restriction fragments;
(b) ligating the restriction fragments thus obtained to an adapter;
(c) amplifying the mixture of adapter-ligated restriction fragments thus obtained with one or more S3P-primers, preferably one S3P primer and an optional second primer, preferably an AFLP-primer to provide a mixture of amplified restriction fragments; and
(d) optionally, detecting at least one of the amplified restriction fragments thus obtained.
   In the above aspects of the invention, the (starting) nucleic acid is preferably a DNA sequence, more preferably a double stranded DNA sequence.

In particular, the starting nucleic acid can be a nucleic acid that contains (or is at least suspected to contain) a splice site to which the S3P-primer used can and/or is intended to hybridise. For instance, the starting nucleic acid sequence can be genomic DNA, and in particular eukaryotic genomic DNA, or (a mixture or a library of) recombinant DNA clones, e. g. derived from a plant, animal or a human. For instance, the starting nucleic acid can be derived from agronomically important crops such as wheat, cucumber, melon, barley, maize, tomato, pepper, lettuce, rice, soybean etc.; from animals such as such as mouse, rat, pig, chicken, fish, etc.; and/or from humans.

In the restriction step a), the starting nucleic acid is restricted with a restriction endonuclease, which may be any suitable restriction endonuclease, such as a Type II or Type IIs, including but not limited to those mentioned below.

In particular, the starting nucleic acid may be restricted with two different restriction endonucleases. For instance, the starting nucleic acid may be restricted with a frequent cutter restriction endonuclease, which serves the purpose of reducing the size of the restriction fragments to a range of sizes that are amplified efficiently; and a rare cutter restriction endonuclease, which serves the purpose of targeting rare sequences. For both, reference is made to for instance EP-A-0 534 858 and EP-A-0 721 987 by applicant, incorporated herein by reference. The skilled person will understand that the recognition sequence of a frequent cutter usually has no more than four bases that provide selectivity, whereas a rare cutter usually has at least six selective bases in its recognition sequence. However, whether a given enzyme functions as a rare or a frequent cutter also depends on the base composition of its recognition site and the overall base composition of the sample DNA to be digested. Thus, a four-cutter with only G's and C's in its recognition site may act as a rare cutter on AT-rich DNA. Therefore, a frequent cutter is understood to be a restriction enzyme that upon restriction of a given sample DNA produces restriction fragments the majority of which is less than 1 kb in length, whereas the majority of fragments produced with a rare cutter is larger than 1 kb in length.

Some non-limiting examples of suitable frequent cutter enzymes are MseI, TaqI, and MboI (Sau3A). Some non-limiting examples of commercially available rare cutters are PstI, HpaII, MspI, ClaI, HhaI, EcoRI, EcoRII, BstBI, HinPl, MaeH, BbvI, PvuH, XmaI, SmaI, NciI, AvaI, HaeII, SalI, Xhol and PvuII, of which EcoRI, PstI, HpaII, MspI, ClaI, EcoRU, BstBI, HinPl and MaeII are preferred. Preferably, restriction enzymes are used that produce sticky ends, to facilitate ligation of adapters. In case a combination of two different restriction enzymes is used, preferably not more than one of them is a blunt cutter. When blunt cutters are used, the adapters to be ligated either are to be modified by the use of a helper oligonucleotides to ligate a single stranded adapter or by the use of double stranded adapters.

After the restriction step a), the restricted fragments thus obtained are ligated to an adapter. This adapter will be essentially the same as the adapter (s) used in conventional AFLP, for which reference is again made to the prior art relating to AFLP mentioned above. As in conventional AFLP, the adapter used is preferably such that it is suitable for use with at least one of the restriction enzymes used in the restriction step a). For instance, when the starting DNA is restricted with two restriction endonucleases (e. g. a frequent cutter and a rare cutter) preferably also two adapters are used, each suitable for use with one of the restriction endonucleases.

The method associated with the present invention can also, be performed using at least one restriction endonuclease, at least one adapter and at least one AFLP primer in combination with an S3P primer. The endonuclease is preferably a frequent cutter.

After the adapter has been ligated to the restriction fragments, the mixture of adapter-ligated restriction fragments thus obtained may then (directly) be amplified in step c) with the S3P-primer and the AFLP-primer. Alternatively, the mixture can be amplified using one or more S3P primers, and/or optionally an AFLP primer. As already indicated above, prior to the amplification step c), the (adapter-ligated) restriction fragments may first be subjected to a pre-amplification using one or two AFLP primers or one or more S3P primer, and in particular a selective pre-amplification for reducing the complexity of the fragment mixture. For instance, such a selective preamplification may be carried out analogous to a selective pre-amplification known per se from AFLP, for which reference is again made to the prior art related to AFLP mentioned above. Alternatively, such a preamplification may be performed suing one or more splice site-specific primers.

More generally, the restriction step a), the ligation step b) and any preamplification step described above may be carried out in essentially the same manner as the restriction, ligation and amplification steps of conventional AFLP methodology, e.g. according to known AFLP protocols. The subsequent amplification step c) of the adapter-ligated restriction fragments with the S3P-primer and the AFLP-primer may be carried out as described hereinabove and as illustrated in FIG. 1, in which the adapter-ligated restriction fragments serve as the target nucleic acid (2).

Thereafter, the amplified mixture thus obtained is analysed, which is also carried out as described hereinabove. Generally, these detection techniques will be such that they allow for the detection of polymorphisms, e. g. detectable signals that are unique for the starting nucleic acid. For instance, such a unique detectable signal may be a unique band in a fingerprint or a unique hybridisation event/signal on an array ; or the lack of such a band or hybridisation signal. For this purpose, the detectable signal (s) generated for a specific starting nucleic acid will usually be compared to the detectable signal (s) obtained for one or more related starting nucleic acid (s) under essentially the same conditions (e. g. the use of the same restriction enzymes, adapters, preamplification (if any), S3P-primer, AFLP-primer and detection technique), for instance by comparing fingerprints and/or hybridisation signals/patterns on a given array. Such related starting nucleic acids may for instance have been derived from the same individuals and/or from one or more closely related individuals (e. g. from the same family, genus, species or even variety). For instance, one or more such related starting nucleic acids may be used/incorporated as reference sample (s) in the method of the invention, in which case the results for the starting nucleic acid sequence and the reference sample (s) may be directly compared. Alternatively, the results obtained for a given starting nucleic acid may be compared to results generated earlier for one or more related nucleic acid sequences, which may for instance be part of a database.

Again, such detection techniques and techniques for analyzing/comparing the results obtained will be essentially analogous to the techniques used to analyze the results obtained using AFLP, for which again reference is made to the prior art related to AFLP mentioned above. Thus, from the above, it will be clear that the method of the invention may conveniently be carried out analogous to a conventional AFLP amplification, in which for the main amplification (as opposed to the pre-amplification) a combination of one AFLP primer and one S3P-primer is used, instead of two AFLP-primers. Alternatively the amplification can be performed using one or more S3P primers, optionally in combination with one or more AFLP primers.

Also, as in conventional AFLP, the selective nucleotides (9) of the AFLP-primer (7) may be selected arbitrarily or randomly. Also, the S3P-primer (1), may comprise, in addition to the nucleotides that are part of the consensus sequence of the splice site, nucleotides (6) that are not part of the consensus sequence of the splice site. These nucleotides can be located adjacent to one or both sides of the consensus sequence or can be located intermittently in the consensus sequence in case the consensus sequence is not consecutive. These nucleotides can be randomly selected or can be purposively selected. When randomly selected, these nucleotides provide the same selective function as the selective nucleotides of the AFLP primers, i.e. the reduction of the number of fragments to be amplified, to create a subset of amplified splice site related adapter ligated restriction fragments. When purposively selected the selective nucleotides provide the selectivity that may be used to specifically select the amplification/detection of a predetermined splice site polymorphism, i.e. a splice site of which not only the sequence is known but also the intermittent or adjacent sequence is identified. The randomly chosen nucleotides in the splice site primer can also be selected such that groups of splice site-specific primers are formed. These effects may also occur due to the natural degeneration of the primers. The S3P primers in such group are selective for a group of specific splice sites that in addition to the consensus sequence comprises a further set of selective nucleotides. This provides for an additional possibility to selectively amplify subsets of splice site related adapter ligated restriction fragments. It is also possible to include non-selective nucleotide analogues such as inosines, and the like to provide for reduced selectivity or to avoid certain degenerate positions in a splice site.

One of the advantages of the present method is that, as with conventional AFLP, the method of the invention does not require any prior knowledge of the sequence to be analysed, nor the use of any specifically designed primers, apart from the splice site-specific part. In addition, the invention may allow the detection of splice site-associated polymorphisms/markers in conjunction with AFLP-markers, and thus provide a very powerful (combined) technique for the analysis of a starting nucleic acid for both these types of highly informative genetic markers. However, as with conventional AFLP, it may be that some (combinations of) specific AFLP-primers and S3P-primers may provide, for a specific starting DNA, more informative results (e.g. more polymorphic fragments) than other combinations, and that some combinations may even provide no informative results at all. Nevertheless, based upon the disclosure herein, the skilled person will be able to provide one or more suitable combinations of a S3P-primer and an AFLP-primer for analyzing a specific starting DNA according to the method of the invention, optionally after some preliminary experiments and/or a limited degree of trial and error.

In principle, the method of the invention can be used for any application for which a splice site associated polymorphic marker can be developed or used. Such applications include, but are not limited to, genotyping, genetic mapping, genetic profiling and DNA-identification techniques, e.g. to identify a specific species, subspecies, variety, cultivar, race or individual, to establish the presence or absence of a specific inheritable trait and/or of a gene; or to determine the state of a disease.

The invention can also be used for removing band patterns from fingerprints that have been caused by amplification of chloroplast sequences, due to the differences between splice site sequences of nuclear coded and plastome genes

Generally the methods of the invention may provide the advantages of:
efficient targeting of a large proportion of splice sites present in the genome;
the provision of more direct information pertaining to coding regions of the genome and consequently of markers that may be more closely linked to genic regions or traits of interest; and
highly reproducible fingerprint patterns due to excellent reproducibility of the AFLP technique compared to other techniques.

According to the invention, one or more of the splice site-associated markers identified-using the method of the invention may be (further) developed into a classical PCR-test. This may for instance be carried out by a method as schematically illustrated in the non-limiting FIG. 4

In one aspect, the present invention accordingly pertains to a method for the determination of PCR-primers, the PCR-primers, preferably determined by the method, the use thereof in the development of a PCR-assay and to the use of (a combination of) one or more S3P primers and at least one AFLP primer in the development of PCR-primers. More in particular, the present invention provides a method for the development of PCR-primers that are suitable for use in a conventional PCR-test.

The present invention provides technologies that allow for the conversion of the splice site associated markers into primers that can be used in a conventional PCR test. The present invention also provides for PCR-primers, based on AFLP technology associated with splice sites. Further, the invention provides for primers that can be used in assays based on PCR technology.

Generally, this method involves the identification of a splice site-associated polymorphic fragment, e.g. as described hereinabove. This polymorphic fragment (11) (e.g. a fragment amplified using the combination of a S3P-primer and an AFLP primer for the first restriction enzyme used for AFLP template preparation and optionally one or more alleles thereof) is then isolated (e.g. cut out of the gel obtained after gelelectrophoresis) and sequenced (step 1 in FIG. 4). Based upon the sequence of the polymorphic fragment (s) thus obtained, a suitable PCR-primer is selected/designed from the sequence flanking the splice site sequence at the 3′ end. Next, this PCR primer, in combination with an AFLP primer corresponding to the second enzyme used for AFLP template preparation is used to amplify a fragment that contains the splice site and the 5′ flanking sequence, which is not included in the polymorphic fragment initially chosen for sequencing (step 2). From this 5′ flanking sequence a suitable second PCR primer is selected/designed (step 3), which together with the first PCR primer matching the 3′flanking sequence is used in a conventional PCR-detection, e. g. on a starting DNA (step 4).

In one aspect, the invention pertains to a method for providing a PCR primer comprising the steps of identification of a splice site-associated polymorphic fragment amplified by the combined use of a S3P primer and an AFLP primer for the first restriction enzyme used for AFLP template preparation, sequencing the fragment, designing and synthesizing a first PCR-primer for the sequence flanking the splice site sequence at the 3′ end; optionally amplifying a fragment comprising the splice site and at least part of the 5′-flanking sequence using the first PCR-primer and a second AFLP primer used for AFLP template preparation, and optionally designing and synthesizing a second PCR-primer for the sequence flanking the splice site sequence at the 5′ end.

The method according to the invention involves the identification of a splice site associated polymorphic fragment, e.g. as described hereinbefore. This polymorphic fragment (e. g. a fragment amplified using the combination of a S3P primer and an AFLP-primer for the first restriction enzyme used for AFLP template preparation and optionally one or more alleles thereof) is isolated (e. g. cut out of the gel obtained after gelelectrophoresis) and sequenced (step 1 in FIG. 4). For sequencing, the gel-excised fragments may be cloned in convenient sequencing vectors. Alternatively, the gel-excised fragments are re-amplified in a PCR using the S3P-primer used in the original amplification, and a modified version of the AFLP primer used in the original amplification. The modified AFLP-primer preferably contains an additional sequence at its 5′-end that may conveniently be used for priming subsequent sequencing reactions. A convenient example of such additional sequence for priming sequencing reactions is sequence of the universal M13 sequencing primer.

Based on the sequence of the polymorphic fragment (s) thus obtained, a suitable PCR primer is selected/designed from the sequence flanking the splice site sequence at the 3′-end. Next, this PCR-primer, in combination with an AFLP-primer corresponding to the second enzyme used for AFLP template preparation, is used for the amplification of a fragment that contains the splice site and an additional 5′flanking sequence that is downstream from the splice site with respect to the first PCR primer. This additional 5′-flanking sequence was not present in the polymorphic band initially chosen for sequencing (step 2 in FIG. 4). The additional 5′-flanking sequence is used as basis for the design of a suitable second PCR-primer (step 3 in FIG. 4 which together with the first PCR primer matching the 3′-flanking sequence is suitable for use in a conventional PCR-detection, e. g. on the starting DNA (step 4 in FIG. 4)

The present invention provides for a reliable and powerful method for the generation of PCR primers. The PCR primers obtained according to the invention preferably are suitable for use in conventional PCR-technology and more preferably are suitable PCR primers for use in conventional assays based on flanking PCR primers, whereby the splice sites have been identified using splice site AFLP technology. Hence, splice site AFLP provides a valuable technique for rapid and reliable identification of polymorphic splice sites. A further advantage is that the possibility is provided for the selective enrichment of nuclear coded sequences or organelle coded sequences by using the 3′end, which is a distinct advantage of the present invention over the conventional techniques.

In a preferred embodiment of the invention, the steps indicated as optional are also included in the method. It is hence preferred that a second PCR primer is designed for the development of an assay based on conventional PCR. The skilled person will appreciate that alternative methods exist for obtaining the additional flanking sequence based on which the second PCR primer will be designed, in addition to the method based on the second AFLP-primer as specifically disclosed in the present application. Such methods e.g. include sequencing of fragments obtained by inverse PCR.

A flanking sequence in terms of the present invention refers to a sequence adjacent to a splice site sequence. The length of a flanking sequence will usually be defined by the distance between a splice site sequence and another sequence, e.g. another splice site-specific sequence or a sequence designated or suitable as PCR-primer or AFLP primer and the like. The length of a flanking sequence generally varies between 0 and 500 nucleotides, preferably up to 250, more preferably up to 150 and most preferably up to I 00 nucleotides. The upper limit will generally be governed by factors such as the resolution of the gel and the length of the splice site derived fragment.

In a further aspect, the invention pertains to a method for the determination of a PCR-primer, comprising the steps of:
restricting a nucleic acid sequence with a restriction endonuclease to provide a mixture of restriction fragments;
ligating the restriction fragments thus obtained to a adapter;
amplifying the mixture of adapter ligated restriction fragments thus obtained with a S3P-primer and a first AFLP primer to provide a mixture of amplified restriction fragments;
detecting at least one of the amplified restriction fragments thus obtained;
identifying a splice site-associated polymorphic fragment or band;
determining the sequence of the polymorphic fragment or band;
designing a first PCR-primer for the sequence flanking the splice site sequence at the 3′end;
optionally amplifying a fragment comprising the splice site and at least part of the 5′-flanking sequence using the first PCR-primer and a second AFLP primer;
optionally designing a second PCR-primer for the sequence flanking the splice site sequence at the 5′end.

The invention further relates to primers obtainable by the present invention in the development of an assay, preferably for the analysis of splice sites.

The invention further relates to the use of (the combination of) a S3P primer and an AFLP primer in the development of PCR-primers, preferably suitable for use in splice site assays.

The polymorphic fragment which is used to determine a suitable PCR-primer is preferably derived from genomic DNA; and in particular eukaryotic genomic DNA or (a mixture or a library of) recombinant DNA clones e.g. derived from a plant, animal or human being.

The invention also relates to the use of a PCR-primer according to the present invention in the development of an assay, preferably for the analysis of splice sites and to a kit comprising means for obtaining a PCR-primer according to the invention, as well as to a kit comprising a PCR-primer according to the invention.

Furthermore, one or more of the splice site-associated polymorphic fragments identified by the method of the invention is isolated and optionally sequenced, and is used to generate a nucleotide sequence representative for the splice site-associated marker for use in—for instance—an array for the analysis of nucleic acid sequences.

In yet another aspect, the invention relates to the use of a S3P-primer in the methods described hereinabove. The invention also relates to the use of an AFLP primer in the methods described hereinabove.

In another aspect, the invention relates to the use of a combination of a S3P primer and an AFLP-primer in analyzing a nucleic acid sequence. In particular, this aspect of the invention relates to the use of the combination of a S3P-primer and a AFLP-primer in analyzing a nucleic acid sequence for the presence of polymorphisms associated with splice sites.

Yet another aspect comprises any data generated by the method of the invention, optionally on a suitable data carrier, such as paper or a computer disk. Such data may for instance include the generated DNA-fingerprints (e. g. in the form of a gel) and/or autoradiographs/photographs or other reproductions thereof, as well as (stored) analogous or digital data thereon, e. g. in the form of a database.

The invention also comprises kits for use in the invention, the kits at least comprising a S3P-primer and an AFLP-primer; and usually also comprising an adapter complementary to the AFLP-primer. These kits can further contain any known component for such kits, including but not limited to components known per se for AFLP kits, such as restriction enzymes (in which case the adapters are preferably suited to be ligated to the restricted sites generated with the enzyme); a polymerase for amplification, such as Taq-polymerase; nucleotides for use in primer extension; as well as buffers and other solutions and reagents; manuals, etc. Further reference is made to the European patent application 0 534 858, incorporated herein by reference.

### DESCRIPTION OF THE FIGURES

FIG. 1 is a schematic representation of the method of the invention. In FIG. 1, 1 depicts a S3P primer, 2 is the double stranded target DNA (restriction fragment), 3 is the splice site, the intron part of the splice site is indicated as 3A, the exon part as 3B, 4 is the part of the S3P primer located at the 3′ end and 5 is the 5′ end. The AFLP primer is (7) contains a part (10) that is complementary to the adapter (8) ligated to the restriction fragment and may contain selective nucleotides at the 3′ end (9)

FIG. 2 and 2A are a exemplary representation of a consensus sequence of a splice site in combination with a target sequence and two primers, one mismatching on the target sequence and one matching primer, thereby introducing selective nucleotides in the S3P Primer. FIG. 2A is an exemplary representation of a consensus sequence of a splice site.

FIG. 3 is an AFLP-fingerprint generated with a splice-site specific primer in combination with an AFLP primer. PCR profile A: 30 s at 94° C.+13*(30 seconds at 65° C., 0.7° C./cycle Touch Down)+60 seconds at 72° C.; 30 s at 94° C.+23*(30 seconds at 50° C.)+60 seconds at 72° C.; PCR profile B: 30 s at 94° C.+13*(30 seconds at 65° C., 0.7° C./cycle Touch Down)+60 seconds at 72° C.; 30 s at 94° C.+23*(30 seconds at 56° C.)+60 seconds at 72° C.; PCR profile C: 30 s at 94° C.+13*(30 seconds at 45° C., 1° C./cycle Touch Up)+60 seconds at 72° C.; 30 s at 94° C.+23*(30 seconds at 50° C.)+60 seconds at 72° C.;

Sections 1-6 are based on +0/+0 AFLP preamplification with primer combination SSPn/AFLP+0. Sections 7-13 are based on +0/+0 AFLP preamplification with primer combination SSPn/AFLP+1. Sections 14-18 are based on +1/+1 AFLP preamplification with primer combination SSPn/AFLP+3.

FIG. 4 is a schematic representation of the conversion into a PCR assay.

FIG. 5 is a representation of a splice-site AFLP screening on Arabidopsis RIL8 and tomato parental line samples. The left panel represents Arabidopsis RIL8 sample screening using a Splice site primer SSP9 and MseI+0 primer (M00k) combination on template generated using EcoRI and MseI. Lane 13 represents the 10bl size marker, lane 14 and 15 represent the parental lines 1 and 2, respectively. The right two panels represent the screening of Splice site primers SSP3 and SSP9 combined with an AseI+1 primer using AseI templates of tomato parental lines.

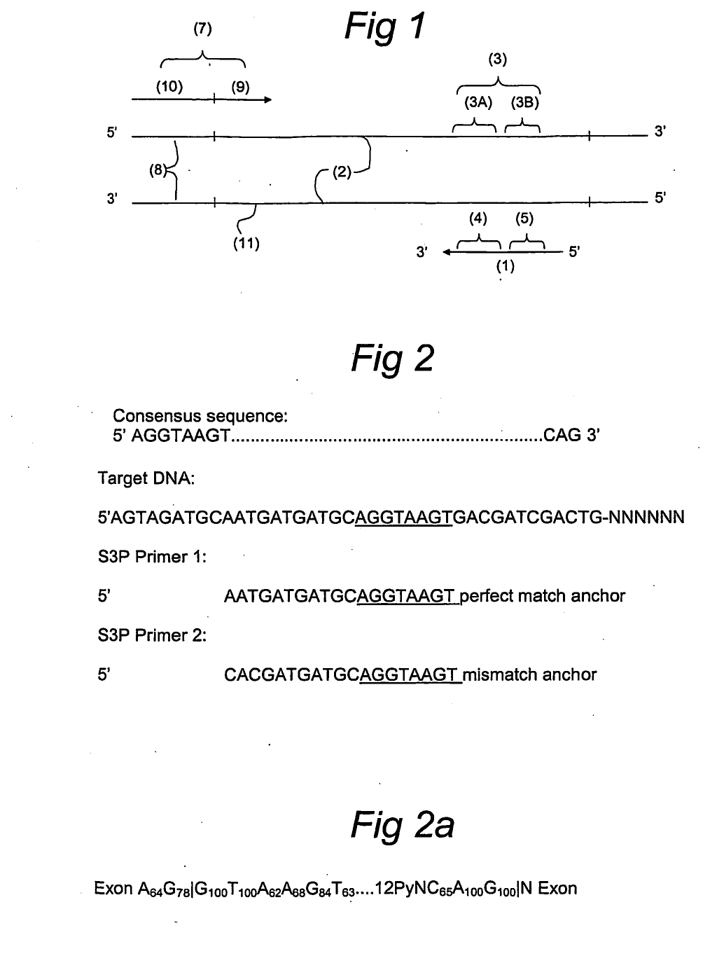

### EXAMPLES

### Example 1

DNA from the Arabidopsis lines Landsberg erecta and Columbia was used to generate AFLP fingerprints by use of a splice-site-specific primer (S3P primer in combination with an +0, +1, +2 or +3 EcoRI or MseI AFLP primer. AFLP-reactions with 12 different splice-site specific primers (Table 3) in combination with 10 different AFLP primers were performed on AFLP restriction-ligation mixture, +0/+0 or +1/+1 AFLP preamplification product.

Three different PCR-profiles were used for the amplification of the fragments. AFLP fragments obtained with were excised out of PAA-gels (56 AFLP-marker bands and 4 constant bands) and reamplified. Twelve markers were cloned by use of the Original TA Cloning Kit (Invitrogen) and 32 clones were sequenced on the MegaBACE. To find out if coding regions are preferentially amplified by Splice-site AFLP, the presence of coding regions in sequences of AFLP fragments obtained with Splice-site AFLP and sequences of Arabidopsis EcoRI/MseI+2/+3 AFLP markers, obtained with the standard AFLP procedure was determined by a BLAST-search, performed with the PEDANT-software of Biomax (Martinsried, Germany).

To avoid sequencing problems that may occur when fragments are excised out of PAA-gels with dense fingerprint patterns because multiple fragments may migrate at the same position and the fragments are not always excised from the PAA-gel without contamination by other fragments, a cloning method was used. By cloning these excised fragments, pure fragments are obtained and sequencing these clones prevents these problems.

Results

Fingerprint Results

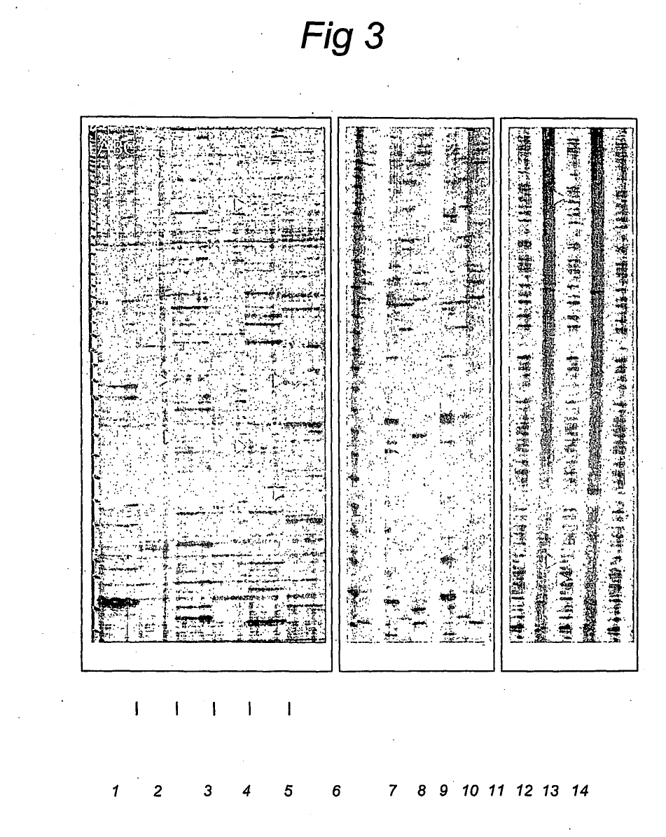

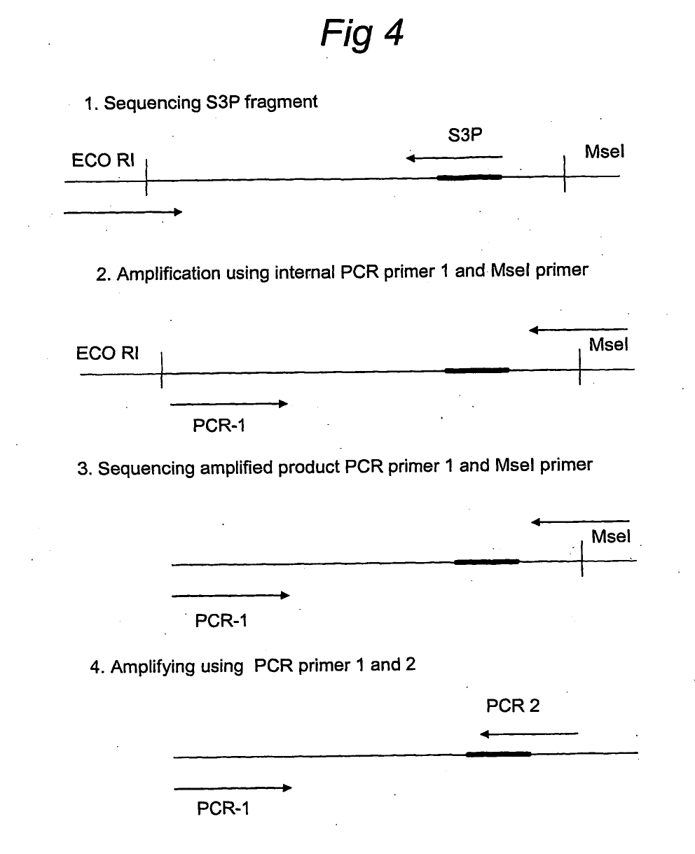

Fingerprints generated by use of a splice-site-specific primer (S3P primer) in combination with a +0, +1, +2 or +3 EcoRI or MseI AFLP primer and the PCR-profiles used for the amplification are shown in FIG. 3. Splice-site AFLP markers are marked by arrows.

PCR profile A: 30 s at 94° C.+13*(30 seconds at 65° C., 0.7° C./cycle Touch Down)+60 seconds at 72° C.; 30 s at 94° C.+23*(30 seconds at 50° C.)+60 seconds at 72° C.; PCR profile B: 30 s at 94° C.+13*(30 seconds at 65° C., 0.7° C./cycle Touch Down)+60 seconds at 72° C.; 30 s at 94° C.+23*(30 seconds at 56° C.)+60 seconds at 72° C.;PCR profile C: 30 s at 94° C.+13*(30 seconds at 45° C., 1° C./cycle Touch Up)+60 seconds at 72° C.; 30 s at 94° C.+23*(30 seconds at 50° C.)+60 seconds at 72° C.;

Sections 1-6 are based on +0/+0 AFLP preamplification with primer combination SSPn/AFLP+0. Sections 7-13 are based on +0/+0 AFLP preamplification with primer combination SSPn/AFLP+1. Sections 14-18 are based on +1/+1 AFLP preamplification with primer combination SSPn/AFLP+3.

Sequence Analysis

After a BLAST search against a public eukaryotic genomic DNA data set, 2 hits with predicted genes were found (28.5%) (Table 2). A BLAST search performed with 132 Arabidopsis AFLP fragment sequences against this eukaryotic genomic DNA data set resulted in 10 hits with predicted genes (7.5%). This demonstrates that coding regions are preferentially amplified.

TABLE 2
Hits of predicted genes found for 2 splice-site AFLP fragments in the BLAST-search:

| Code | Contig | Start | Stop | Description | Best BLAST hit | Hit ID | e-val |
|---|---|---|---|---|---|---|---|
| gene1 | G1 F508.850 | 95 | 280 | Predicted gene | Hypothetical protein F23A5.3 | PIR: B96839 | 9e−32 |
| | | | | | [imported] - Arabidopsis thaliana | | |
| gene1 | E1 F524.480 | 372 | 12 | Predicted gene | Phosphatidylinositol 3-kinase | PIR: B96630 | 2e−47 |
| | | | | | [imported] - Arabidopsis thaliana | | |

The concept of generating AFLP markers by use of a splice-site specific primer in combination with an AFLP primer works. Markers were detectable when Splice-site AFLP was performed on AFLP preamplification product. Using splice-site specific primers in combination with +0 AFLP primers and +0/+0 AFLP preamplification product, dense fingerprints were generated. Five different splice-site specific primers used in combination with one +3 AFLP primer and +1/+1 preamplification product resulted in completely reproducible fingerprints. This indicates that targeting of the splice-site specific primer is based on the splice-site specific sequence. The degree of polymorphism was comparable with a standard +2/+3 AFLP fingerprint (14%). The three evaluated PCR profiles produced nearly the same fingerprints.

### Example 2

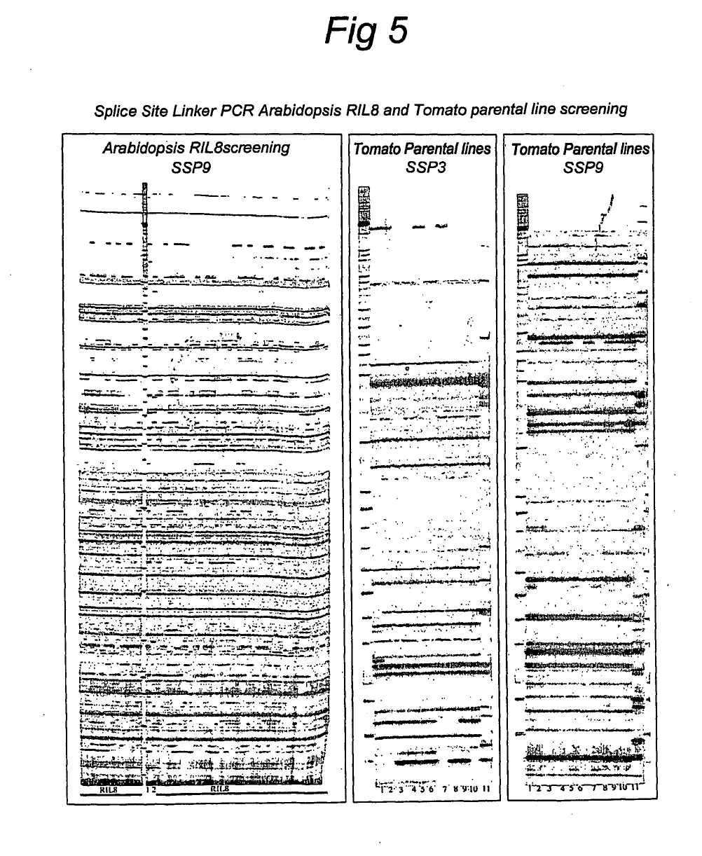

In this Example, it was the objective to enrich fingerprints for genic regions (intron or exon sequences) of the genome on a larger scale than in example 1. The targeting efficiency was determined by sequencing of splice-site PCR fragments followed by homology searches. Furthermore, several splice-site PCR primers were tested on tomato parental lines. The 12 selected and designed splice-site primers from the previous example were used to determine the optimal primer/enzyme combination and the optimal amplification profile. They were designed on Arabidopsis sequences but were also usable to generate fingerprints in tomato. An example of fingerprints generated using splice-site primers on Arabidopsis and tomato is shown in FIG. 5. This example clearly shows the segregation of markers in the RIL8 population. Furthermore, it shows that the splice-site primers can be used to generate reproducible fingerprints in tomato.

To determine the targeting efficiency in tomato splice-site PCR fragments were isolated from polyacrylamide gels with fingerprints of tomato samples using the SSP1, SSP3 or SSP9 primer and subsequently sequenced. From these sequences, 53 sequences of good quality were used for homology analysis, which rendered 6 sequences with homology to protein sequences, which is 11.3% of the fragments. To compare the targeting efficiency of genic regions using regular AFLP, fragments from polyacrylamide gels with fingerprints of tomato samples generated using regular AFLP, were excised, reamplified and sequenced. A total of 62 sequences of good quality were used for homology analysis, which rendered 3 sequences with homology to protein sequences, which is 4.8% of the fragments. Compared to this 4.8% targeting efficiency of genic regions using regular AFLP, the targeting efficiency is raised 2-3 fold in tomato by using splice-site linker PCR. New primers were designed using sequences of splice-site PCR fragments that rendered a homology with proteins from the database. These primers where designed to be directed into the intron of the protein, whereas the splice-site PCR fragment sequences were directed into the exon. These new SSP primers also generated fingerprints that could be readily used for genotyping. A total overview of available and tested splice-site primers is shown in Table 3.

TABLE 3
overview of splice-site primers

| | | PCR | | | | |
|---|---|---|---|---|---|---|
| SEQ ID | | primer | Primer Sequence | | Splice-site | |
| # | SSP nr | nr | 5′-3′ | Orientation | fragment | Remarks |

| | | | | | | |
|---|---|---|---|---|---|---|
| 1 | SSP 1 | 01S037 | CATGCATGACACTTACCTG | Intron -> Exon | | |
| 2 | SSP 2 | 01S038 | CTCGATGTATGACTTACCTK | Intron -> Exon | | |
| 3 | SSP 3 | 01S039 | GATTCACGGCASACTTACCT | Intron -> Exon | | |
| 4 | SSP 4 | 01S040 | GAGACTGTASACTTACCTG | Intron -> Exon | | |
| 5 | SSP 5 | 01S041 | GACTGATAAGCGACTTACCT | Intron -> Exon | | |
| 6 | SSP 6 | 01S042 | CTGATAGCTCACTTACCT | Intron -> Exon | | |
| 7 | SSP 7 | 01S043 | CTCGATTCAGACTTACCTGA | Intron -> Exon | | |
| 8 | SSP 8 | 01S044 | TTTTTTTTTTTTTTTTGCAGGTTG | Intron -> Exon | | |
| 9 | SSP 9 | 01S045 | TTTTTTTTTTTTTTTTGCAGGTAG | Intron -> Exon | | |
| 10 | SSP 10 | 01S046 | TTTTTTTTTTTTTTTTTTGCAGGT | Intron -> Exon | | |
| 11 | SSP 11 | 01S047 | CTGATAGNNNACTTACCT | Intron -> Exon | | |
| 12 | SSP 12 | 01S048 | TTTTTTTTTTTTTTTTTTTRCAGR | Intron -> Exon | | |
| 13 | SSP 13 | 02Y053 | AAATCGTTTTTCCAGGTAAG | Exon -> Intron | C04/05 | SSP1 fragment |
| 14 | SSP 14 | 02Y054 | ATCTCCATTCGGCAGGTAAG | Exon -> Intron | D11 | SSP1 fragment |
| 15 | SSP 15 | 02Y055 | GATCTTCGGAAGCAGGTAAG | Exon -> Intron | E05 | SSP1 fragment |
| 16 | SSP 16 | 02Y056 | ATGTCGTCAATGCAGGTAAG | Exon -> Intron | E10 | SSP1 fragment |
| 17 | SSP 17 | 02Y057 | TGTCTCTGAGTGCAGGTAAG | Exon -> Intron | F04/05 | SSP1 fragment |
| 18 | SSP 18 | 02Y058 | CTCAGAATTTTCCAGGTAAG | Exon -> Intron | F08 | SSP1 fragment |
| 19 | SSP 19 | 02Y059 | AAGAAAACACAGCAGGTAAG | Exon -> Intron | F09 | SSP1 fragment |
| 20 | SSP 20 | 02Y060 | TCGAATTGTCACCTAACCTG | Exon -> Intron | G01 | SSP9 fragment |
| 21 | SSP 21 | 02Y061 | TCGCTGCACTCCTTAACCTG | Exon -> Intron | G02 | SSP9 fragment |
| 22 | SSP1B | 02Y062 | CGTCATGCATGACACTTAC | Intron -> Exon | | SSP1 - 3′CTG |
| 23 | SSP5B | 02Y063 | CTGACTGATAAGCGACTTAC | Intron -> Exon | | SSP5 - 3′CT |
| 24 | SSP7B | 02Y064 | CTGACTCGATTCAGACTTAC | Intron -> Exon | | SSP7 - 3′CTGA |
| 25 | M00k | | GATGAGTCCTGAGTAA | | | |

1-25. (canceled)

26. A method for analyzing or amplifying a nucleic acid sequence, comprising analyzing or amplifying a nucleic acid with an S3P primer.

27. The method according to claim 26, wherein said S3P primer is in combination with at least one AFLP primer.

28. The method according to claim 26, wherein the nucleic acid sequence comprises a restriction fragment to which one adapter sequence has been ligated.

29. The method according to claim 28, in which the restriction fragment to which the adapter sequence has been ligated is part of a mixture of adapter-ligated restriction fragments.

30. The method according to claim 26, in which the nucleic acid sequence contains or is suspected to contain, an intron-exon junction and/or a splice site.

31. The method according to claim 26, in which the restriction fragment is derived from genomic DNA, mitochondrial DNA, chloroplast DNA, recombinant DNA or unprocessed heteronuclear mRNA.

32. The method according to claim 26, wherein the S3P primer is in an intron-to-exon orientation or in an exon-to-intron orientation.

33. The method according to claim 27, wherein the AFLP primer contains at least one selective nucleotide at its 3′ end.

34. The method according to claim 26, wherein the S3P primer comprises a conserved splice site border sequence or at least part of a consensus sequence.

35. The method according to claim 34, wherein the S3P primer further comprises a random sequence.

36. The method according to claim 26, wherein S3P primer is specific for a splice site selected from the group consisting of GU-AG introns, AU-AC introns, Group I introns, Group II introns, Group III introns, Twintrons, Pre-tRNA introns, and splice sites that are identified using computer based splice site identification methods.

37. The method according to claim 26, wherein the S3P primer contains a total of between 8 and 20 nucleotides.

38. The method according to claim 26, wherein between 4 and 10 nucleotides present in the S3P primer are complementary to the conserved region or consensus sequence of the splice site.

39. The method according to claim 26, wherein the consensus sequence is X₁X₂GTX₃X₄X₅X₆, wherein X₁, X₂, X₃, X₄, X₅, X₆ are independently selected from the group consisting of A, C, T, or G.

40. The method according to claim 39, wherein the consensus sequence is AGGTAAGT.

41. A method for analyzing a nucleic acid sequence, comprising:
(a) amplifying an adapter-ligated restriction fragment generated from the nucleic acid to be analysed, using one or more S3P-primers and optionally an AFLP-primer to amplify the nucleic acid sequence; and optionally comprising the further step of:
(b) detecting the amplified nucleic acid sequences thus obtained.

42. A method for analyzing a nucleic acid sequence, the method comprising the steps of:
(a) restricting the starting nucleic acid with a restriction endonuclease to provide a mixture of restriction fragments;
(b) ligating the restriction fragments thus obtained to at least one adapter;
(c) amplifying the mixture of adapter-ligated restriction fragments thus obtained with one or more S3P-primers and optionally at least one AFLP-primer to provide a mixture of amplified restriction fragments; and optionally comprising the further step of
(d) detecting the amplified restriction fragments thus obtained.

43. A method for the amplification of at least one restriction fragment obtained from a starting DNA, comprising:
(a) digesting the starting DNA with at least one restriction endonuclease, thereby providing one or more restriction fragments;
(b) ligating at least one oligonucleotide adapter to one or both ends of the restriction fragments to provide adapter-ligated restriction fragments;
(c) providing a primer set comprising one or more S3P primers and optionally at least one AFLP primer;
(d) contacting the adapter-ligated restriction fragments with the set of primers;
(e) amplifying the adapter-ligated restriction fragments with the set of primers; and
(f) recovery of any amplified DNA fragments.

44. A method for providing a PCR primer or a pair of PCR primers for use in the amplification of a PCR fragment spanning a splice site-associated genomic polymorphism, comprising:
a) identification of a fragment containing the splice site-associated genomic polymorphism, whereby the fragment is amplified by the combined use of one or more S3P primers and optionally at least one first AFLP primer for a first restriction enzyme used for AFLP template preparation;
b) sequencing the polymorphic fragment;
c) synthesizing a first PCR-primer corresponding to a sequence flanking the splice site sequence at the 3′ end;
d) optionally, amplifying a fragment comprising the splice site-associated genomic polymorphism and sequences flanking the splice site-associated genomic polymorphism at its 5′-end, using the first PCR-primer and a second AFLP primer for a second restriction enzyme used for AFLP template preparation; and,
e) optionally, synthesizing a second PCR-primer corresponding to a sequence flanking the splice site sequence at the 5′end.

45. A method for providing a PCR-primer, comprising:
a) restricting a nucleic acid sequence with at least one restriction endonuclease to provide a mixture of restriction fragments;
b) ligating the restriction fragments thus obtained to at least one adapter;
c) amplifying the mixture of adapter ligated restriction fragments thus obtained with at least one S3P primer and optionally at least one first AFLP-primer to provide a mixture of amplified restriction fragments;
d) detecting at least one of the amplified restriction fragments thus obtained;
e) identifying at least one splice site-associated polymorphic fragment;
f) determining the sequence of said polymorphic fragment;
g) synthesizing a first PCR-primer corresponding to a sequence flanking the splice site sequence at the 3′ end;
h) optionally, amplifying a fragment comprising the splice site and at least part of the 5′-flanking sequence using the first PCR-primer and a second AFLP primer used in AFLP template preparation; and
i) optionally, synthesizing a first PCR-primer corresponding to a sequence flanking the splice site sequence at the 5′end.

46. A kit comprising at least one S3P primer and optionally at least one AFLP primer.

47. A kit comprising PCR-primers obtained by a method according to claim 44.

48. A method for the enrichment of a sample for nuclear or organelle derived amplification products, comprising enriching the sample according to a method according to claim 43.

49. The method according to claim 41, wherein the S3P-primer is specific for a splice site selected from the group consisting of GU-AG introns, AU-AC introns, Group I introns, Group II introns, Group III introns, Twintrons, Pre-tRNA introns, and splice sites that are identified using computer based splice site identification methods.

50. The method according to claim 42, wherein the S3P-primer is specific for a splice site selected from the group consisting of GU-AG introns, AU-AC introns, Group I introns, Group II introns, Group III introns, Twintrons, Pre-tRNA introns, and splice sites that are identified using computer based splice site identification methods.

51. The method according to claim 43, wherein the S3P-primer is specific for a splice site selected from the group consisting of GU-AG introns, AU-AC introns, Group I introns, Group II introns, Group III introns, Twintrons, Pre-tRNA introns, and splice sites that are identified using computer based splice site identification methods.

52. The method according to claim 44, wherein the S3P-primer is specific for a splice site selected from the group consisting of GU-AG introns, AU-AC introns, Group I introns, Group II introns, Group III introns, Twintrons, Pre-tRNA introns, and splice sites that are identified using computer based splice site identification methods.

53. The method according to claim 45, wherein the S3P-primer is specific for a splice site selected from the group consisting of GU-AG introns, AU-AC introns, Group I introns, Group II introns, Group III introns, Twintrons, Pre-tRNA introns, and splice sites that are identified using computer based splice site identification methods.

54. A kit comprising PCR-primers obtained by the method according to claim 45.


SEQUENCE LISTING (28 sequences)

SEQ ID NO: 1 - DNA, 19 bp, Artificial Sequence
  Description of Artificial Sequence Synthetic primer
catgcatgacacttacctg

SEQ ID NO: 2 - DNA, 20 bp, Artificial Sequence
  Description of Artificial Sequence Synthetic primer
ctcgatgtatgacttacctk

SEQ ID NO: 3 - DNA, 20 bp, Artificial Sequence
  Description of Artificial Sequence Synthetic primer
gattcacggcasacttacct

SEQ ID NO: 4 - DNA, 19 bp, Artificial Sequence
  Description of Artificial Sequence Synthetic primer
gagactgtasacttacctg

SEQ ID NO: 5 - DNA, 20 bp, Artificial Sequence
  Description of Artificial Sequence Synthetic primer
gactgataagcgacttacct

SEQ ID NO: 6 - DNA, 18 bp, Artificial Sequence
  Description of Artificial Sequence Synthetic primer
ctgatagctcacttacct

SEQ ID NO: 7 - DNA, 20 bp, Artificial Sequence
  Description of Artificial Sequence Synthetic primer
ctcgattcagacttacctga

SEQ ID NO: 8 - DNA, 24 bp, Artificial Sequence
  Description of Artificial Sequence Synthetic primer
ttttttttttttttttgcaggttg

SEQ ID NO: 9 - DNA, 24 bp, Artificial Sequence
  Description of Artificial Sequence Synthetic primer
ttttttttttttttttgcaggtag

SEQ ID NO: 10 - DNA, 24 bp, Artificial Sequence
  Description of Artificial Sequence Synthetic primer
ttttttttttttttttttgcaggt

SEQ ID NO: 11 - DNA, 18 bp, Artificial Sequence
  Description of Artificial Sequence Synthetic primer
ctgatagnnnacttacct

SEQ ID NO: 12 - DNA, 23 bp, Artificial Sequence
  Description of Artificial Sequence Synthetic primer
ttttttttttttttttttrcagr

SEQ ID NO: 13 - DNA, 20 bp, Artificial Sequence
  Description of Artificial Sequence Synthetic primer
aaatcgtttttccaggtaag

SEQ ID NO: 14 - DNA, 20 bp, Artificial Sequence
  Description of Artificial Sequence Synthetic primer
atctccattcggcaggtaag

SEQ ID NO: 15 - DNA, 20 bp, Artificial Sequence
  Description of Artificial Sequence Synthetic primer
gatcttcggaagcaggtaag

SEQ ID NO: 16 - DNA, 20 bp, Artificial Sequence
  Description of Artificial Sequence Synthetic primer
atgtcgtcaatgcaggtaag

SEQ ID NO: 17 - DNA, 20 bp, Artificial Sequence
  Description of Artificial Sequence Synthetic primer
tgtctctgagtgcaggtaag

SEQ ID NO: 18 - DNA, 20 bp, Artificial Sequence
  Description of Artificial Sequence Synthetic primer
ctcagaattttccaggtaag

SEQ ID NO: 19 - DNA, 20 bp, Artificial Sequence
  Description of Artificial Sequence Synthetic primer
aagaaaacacagcaggtaag

SEQ ID NO: 20 - DNA, 20 bp, Artificial Sequence
  Description of Artificial Sequence Synthetic primer
tcgaattgtcacctaacctg

SEQ ID NO: 21 - DNA, 20 bp, Artificial Sequence
  Description of Artificial Sequence Synthetic primer
tcgctgcactccttaacctg

SEQ ID NO: 22 - DNA, 19 bp, Artificial Sequence
  Description of Artificial Sequence Synthetic primer
cgtcatgcatgacacttac

SEQ ID NO: 23 - DNA, 20 bp, Artificial Sequence
  Description of Artificial Sequence Synthetic primer
ctgactgataagcgacttac

SEQ ID NO: 24 - DNA, 20 bp, Artificial Sequence
  Description of Artificial Sequence Synthetic primer
ctgactcgattcagacttac

SEQ ID NO: 25 - DNA, 16 bp, Artificial Sequence
  Description of Artificial Sequence Synthetic primer
gatgagtcctgagtaa

SEQ ID NO: 26 - DNA, 46 bp, Artificial Sequence
  Description of Artificial Sequence Synthetic oligonucleotide
agtagatgcaatgatgatgcaggtaagtgacgatcgactgnnnnnn

SEQ ID NO: 27 - DNA, 19 bp, Artificial Sequence
  Description of Artificial Sequence Synthetic primer
aatgatgatgcaggtaagt

SEQ ID NO: 28 - DNA, 19 bp, Artificial Sequence
  Description of Artificial Sequence Synthetic primer
cacgatgatgcaggtaagt